(19) United States
(12) Patent Application Publication
(10) Pub. No.: US 2009/0203776 A1
(43) Pub. Date: Aug. 13, 2009

(54) METHODS OF USING MENTHOL PROPYLENEGLYCOL-CARBONATE AND ANALOGS THEREOF FOR PRODUCING ANTI-INFLAMMATORY AND ANTI-ANGIOGENIC EFFECTS

(76) Inventor: Jonathan R. Matias, Hollis, NY (US)

Correspondence Address: BROOKS KUSHMAN P.C., 1000 TOWN CENTER, TWENTY-SECOND FLOOR, SOUTHFIELD, MI, 48075, US

(21) Appl. No.: 11/575,888

(22) PCT Filed: Sep. 23, 2005
(86) PCT No.: PCT/US05/34399

§ 371 Date: Mar. 23, 2007

Related U.S. Application Data
(60) Provisional application No. 60/612,351, filed on Sep. 23, 2004.

(51) Int. Cl. A61K 31/265; A61K 31/047; A61P 29/00

(52) U.S. Cl. 514/512; 514/738

(57) ABSTRACT

Menthol propyleneglycal-carbonate, analogs thereof and compositions containing such compounds are administered to mammals, preferably humans, to produce anti-inflammatory or anti-angiogenic effects.

### BACKGROUND OF THE INVENTION

The present invention relates to the use of menthol propyleneglycol-carbonate and analogs thereof as anti-inflammatory or anti-angiogenic agents.

Menthol is a natural product which is obtainable from peppermint oil and other mint oils. Menthol and various analogs thereof, such as (−)-isopulegol, N-ethyl-p-menthane-3-carboxyamide and p-methane-3,8 diol, are used in commerce as cooling agents. These compounds impart a cooling sensation to a variety of products, for example, cosmetics, perfumes, personal care products, oral hygiene products, confectionary, cigarettes, cough drops, nasal inhalants and the like. See, also U.S. Pat. No. 5,703,123 to Pelzer, et al.

Menthol has also been applied as a topical antipruitic, and in veterinary medicine as a mild local anesthetic and antiseptic, as well as an internal carminative and gastric sedative. See also, U.S. Pat. No. 5,124,320 to Ivy et al.

Menthol and various of its analogs have also been found to possess anti-fouling activity. See published International Patent Application No. PCT/01/40929.

Menthol has been disclosed as one of several components of a miticide in JP 4305505A.

Research into the use of naturally-occurring chemical compounds for applications as topical medications has been motivated, at least in part, by a growing public concern over the possible health risks associated with products of this type that contain synthetic active agents. Consequently, efforts continue toward the development of safe and effective therapeutic agents based on natural compounds.

### SUMMARY OF THE INVENTION

It has now been discovered, in accordance with the present invention, that carbonic acid derivatives of menthol and various analogs thereof exhibit anti-inflammatory and anti-angiogenic activity.

This discovery is put to practical advantage in the methods of this invention, in which a carbonic acid derivative of menthol or an analog thereof, as described below, is administered to a mammal in an amount sufficient to produce an anti-inflammatory effect or an anti-angiogenic effect in said mammal. The active agent which produces such effect is a compound of the formula:

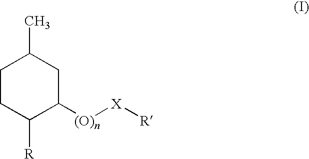

wherein R represents a straight or branched chain, substituted or unsubstituted lower alkyl radical, or a straight or branched chain, substituted or unsubstituted lower alkenyl radical;
X represents a carbonyl linking group (—C(═O)—) or a valence bond;

### n is O or 1; and

R′ represents a radical selected from the group consisting of substituted or unsubstituted hydroxyalkyloxy and substituted or unsubstituted hydroxyalkyl, when n is 1; and R′ represents an alkylamine radical when n is O.

As will appear in the detailed description that follows, compounds of Formula I, above, are effective anti-inflammatory and anti-angiogenic agents.

In published International Application PCT/IN02/00228 menthol propyleneglycol-carbonate and menthol ethyleneglycol-carbonate are disclosed as ingredients of a “cooling cum moisturizing agent” for use as an optional component in an anti-itch formulation. Insofar as is known, however, the compounds described herein as being useful for the practice of this invention have not previously been disclosed or suggested as having anti-inflammatory or anti-angiogenic activities.

### BRIEF DESCRIPTION OF THE DRAWINGS

The sole FIGURE is a set of photographs showing the degree of angiogenic sprouting inside each of 5 aortic rings at various concentrations of racemic methol propylene glycol-carbonate.

### DETAILED DESCRIPTION OF THE INVENTION

Compounds used in the method of this invention are available from commercial sources, including Symrise GmbH and Takasago International USA, among others. The menthol carbonate derivatives may, if desired, be prepared from readily available starting materials, in the manner described in U.S. Pat. No. 5,703,123 to Pelzer, et al. and U.S. Pat. No. 3,419,543 to Mold, et al.

The following definitions apply with reference to compounds encompassed by Formula I, above:

The term “alkyl” refers to straight- or branched-chain unsubstituted alaphatic hydrocarbon groups of 1-12 carbon atoms. Similarly, the term “alkyl”, when used in combination form to name a substituent such as “hydroxyalkyloxy”, “hydroxyalkyl”, “alkylamine” or the like, refers to a straight- or branched-chain alaphatic hydrocarbon group of 1-12 carbon atoms. The expression “lower alkyl” refers to unsubstituted, straight- or branched-chain alkyl groups of 1-6 carbon atoms.

The term “substituted alkyl” refers to an alkyl group substituted by, for example, 1-25 substituents, and most preferably 1-4 substituents. Substituents may include, without limitation, hydroxy, alkoxy, halo, cycloalkoxy, oxo, amino, monoalkylamino, dialkylamino, aryl and substituted aryl. Among the alkyl substituents noted above, particularly preferred are hydroxy substituents.

The term “lower alkenyl” refers to straight- or branched-chain, unsubstituted, unsaturated hydrocarbon groups of 1-6 carbon atoms. Examples of lower alkenyl groups include ethenyl, propenyl, butenyl, pentenyl and the like.

The term “substituted alkenyl” refers to an alkenyl group substituted by, for example, 1-12 substituents, and most preferably, 1-4 substituents. The substituents are the same as those described above with reference to the alkyl groups.

The term “aryl” refers to monocyclic or polycyclic aromatic hydrocarbon groups having 6-15 carbon atoms in the ring portion, such as phenyl, naphthyl, biphenyl, indenyl, fluorenyl or the like, each of which may be substituted.

The term “substituted aryl” refers to an aryl group, as defined above, substituted by, for example, 1-7 substituents, and preferably, 1-4 substituents, such as those described above with reference to the substituted alkyl and alkenyl groups.

The term “halogen” refers to fluorine, chlorine, bromine or iodine.

When a moiety is described herein as substituted with more than one substituent, it is intended that each of the multiple substituents be chosen independently from among the substituents mentioned above.

Compounds encompassed by Formula I, above, have asymmetric carbon atoms, and therefore, can exist as paired enantiomers, differing in their optical activity. The compounds may be used in enantiomerically pure form, in racemic form or in other mixed forms.

Preferred compounds for use in the methods of this invention include: menthol propyleneglycol-carbonate, isopulegol propyleneglycol-carbonate, menthyl-9-hydroxynonyl-carbonate, menthoxy-propane-1,2-diol, and N-ethyl-p-menthane-3-carboxamide.

In carrying out the methods of the invention, compounds of Formula I may be used neat, or as a component of a composition or preparation obtained by admixture with a suitable carrier or vehicle. The nature of the carrier or vehicle will depend on the end use of the composition, i.e. the effect sought to be produced, and the mode of administration.

Compounds of Formula I are preferably formulated with a pharmaceutically acceptable carrier material for administration as an anti-inflammatory agent. A safe and effective amount of the active component for this application is from about 1 wt % to about 30 wt % based on the total weight of the formulation. Satisfactory anti-inflammatory effects have been obtained using menthoxy-propane-1,2-diol, racemic menthol propyleneglycol-carbonate, isopulegol propyleneglycol-carbonate and menthyl-9-hydroxynonyl carbonate.

When used in an anti-angiogenic formulation, a safe and effective amount of the compound of Formula I is in the range from about 1 wt % to about 80 wt % based on the total weight of the composition. Satisfactory anti-angiogenic effect has been obtained using racemic menthol propyleneglycol-carbonate.

The specific amount of compound of Formula I to be used as an anti-inflammatory agent or anti-angiogenic agent may vary depending on differences in potency among the compounds encompassed by Formula I.

In formulations for therapeutic applications, as described above, the formulation may contain one or more compounds of Formula I, above, as the active agent, and, optionally, at least one supplemental active agent. The supplemental active agents may include other anti-inflammatory agents, other anti-angiogenic agents, analgesic agents, antibacterial agents, antiviral agents, antifungal agents, antiparasitic agents, tumoricidal or anti-cancer agents, toxins, enzymes, hormones, neurotransmitters, immunoglobulins, immunomodulators, local anesthetics or the like.

Various auxiliary ingredients may be added to the above-described compositions to impart desired properties or characteristics thereto or to facilitate administration in a particular way. These auxiliary ingredients may include, without limitation, fragrances, surfactants, propellants, emulsifiers, dispersants, buffers, preservatives, antioxidants, diluents, solvents, fixatives, pharmaceutical excipients and adjuvants, as is common practice in the art.

The deleterious activity of microorganisms may be inhibited by the inclusion of various antibacterial and antifungal agents, e.g., paraben, chlorobutanol, phenol, sorbic acid and the like.

The compositions described above may be prepared in various forms depending on the mode of administration. Thus, compositions may be in the form of a lotion, cream, ointment, gel or powder for topical application or a solution or suspension for administration as an atomized or aerosol spray. Alternatively, the composition may, if desired, be in the form of tablets, capsules or microparticulates filled into gelatin capsules, or the like, for oral administration. The composition may also be formed as a suppository for rectal or vaginal administration.

The compounds and compositions described herein may be formulated with sustained release components or carriers of various types, e.g. in alcohol or in water-based formulations for topical use, as is well known in the art.

Compositions used in practicing the invention can be prepared by various methods well known in the art. Typically, such compositions are prepared by intimately mixing a compound of Formula I with a suitable carrier material and optionally one or more supplemental active agents of auxiliary ingredients, as desired, and putting the resulting mixture into a suitable container or dispenser.

The compounds and compositions described herein may be administered systemically or locally, e.g. by application to inflamed areas of skin or by rectal or vaginal delivery. The expression “systemic administration” refers to delivery of an active agent such that it enters the recipient's system and thus, is subject to metabolic processes. Systemic administration encompasses both enteral and parenteral administration, the latter including, without limitation, intravenous, intramuscular, intramedullary, intraperitonal and subcutaneous administration, as well as administration by inhalation.

The compounds and compositions described herein are beneficially administered to mammals, particularly to humans, to produce the desired anti-inflammatory or anti-angiogenic effect.

The following examples set forth further details regarding the invention. These examples are provided for illustrative purposes only, and are not intended to limit the invention in any way. These examples show the results of tests conducted to determine the efficacy of certain compounds of Formula I, above, as anti-inflammatory agents and anti-angiogenic agents.

Example 1 shows the anti-inflammatory effect of compounds of Formula I, above.

### Example 1

Edema was induced in mice by topical application of 10 μl of TPA (Tetradecnoylphorbol acetate) in acetone (2.5 μg/ear) to both the inner and outer surface of one ear of each mouse used in the experiment. Each test compound, diluted with acetone to a concentration of 10% was applied topically to the inflamed mouse ear immediately after TPA application, so as to deliver 2.5 mg/ear. The reference drug, indomethacin (0.5 mg/ear), was administered as a positive control. The thickness of each ear was measured before treatment and 4 hours after induction of inflammation, using a micrometer (Mitutoyo Co.). Anti-inflammatory effect was expressed as the reduction in ear thickness with respect to the control group. The results obtained are presented in Table I.

TABLE I

| | Anti-inflammatory Effect |
|---|---|
| CHEMICAL NAME | (% of control) |
| (l)-Menthol | 0 |
| menthoxy-propane-1,2-diol | 27 |
| N-Ethyl-p-menthane-3-carboxyamide | 11 |
| menthol propyleneglycol-carbonate | 27 |
| racemic menthol propyleneglycol-carbonate | 23 |
| isopulegol propyleneglycol-carbonate | 33 |
| menthyl-9-hydroxynonyl-carbonate | 45 |
| Indomethacin | 96 |

The data show that all of the compounds of the invention that were tested exhibited substantial inhibition of TPA-induced inflammatory response, in comparison to menthol, which showed no appreciable anti-inflammatory effect under the test conditions employed.

Example 2 shows the anti-angiogenic effect of racemic menthol propyleneglycol-carbonate.

### Example 2

The effect of racemic menthol propyleneglycol-carbonate on angiogenesis was studied by culturing aortic explants in three-dimensional matrix gels according to the procedure of Kruger and Figg (Kruger E. A. and Figg, W. D. Protein Binding Alters the Activity of Suramin, Carboxyamidotriazole, and UCN-01 in an ex Vivo Rat Aortic Ring Angiogenesis Assay Clinical Cancer Research 7:1867-1872, 2001).

Thoracic aortas were excised from 8-week-old mail Sprague Dawley rats and the fibroadipose tissue was removed. The aortas were sectioned into 1-mm-long cross-sections, rinsed with Human Endothelial-SFM (GIBCO), placed on the Matrigel-coated wells, covered with additional 50 μl Matrigel, and allowed to gel for more than 30 min at 37° C., 5% CO₂. All the rings were cultured in Human Endothelial-SFM (GIBCO) supplemented with 200 μl/ml of ECGS (Endothelial Cell Growth Supplement, Sigma) as an angiogenesis inducer. Racemic menthol propylene-carbonate diluted with enthanol was added to the culture medium at final concentrations of 1, 10, and 100 μM. Ethanol alone (1%) was added to the culture medium as a vehicle control.

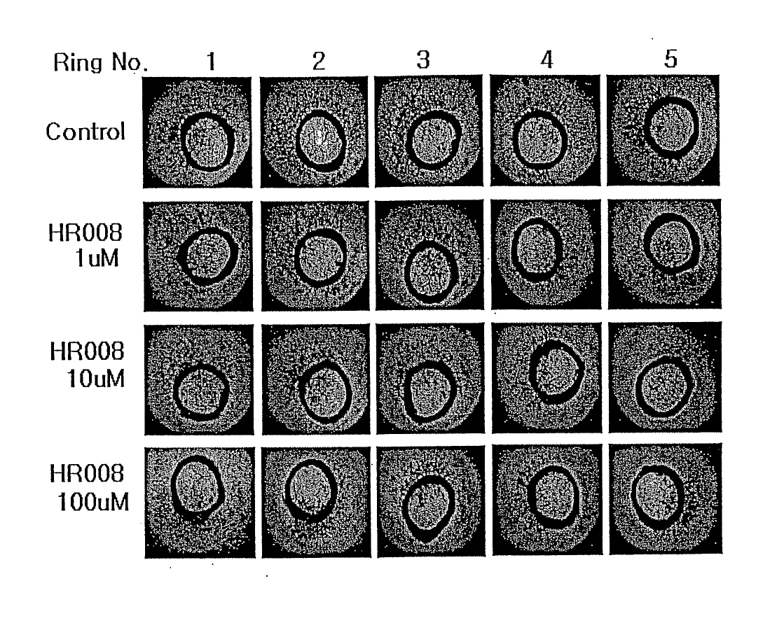

All assays were performed by using 5 aortic rings per sample. Aortic rings were photographed on day 10. These photographs are shown in FIG. 2. The area of angiogenic sprouting was calculated using Image-Pro Plus software (Media Cybernetics). The image analysis quantitation of the dose-response activity of racemic menthol propyleneglycol-carbonate is shown in Table II. Microvessel densities are reported in square pixels.

TABLE II

| | Microvessel Density | |
|---|---|---|
| Concentration (uM) | (pixel²) | % Inhibition |
| 0 | 15.8 ± 4.0 | 0 |
| 1 | 13.4 ± 4.1 | 15 |
| 10 | 12.2 ± 2.5 | 22 |
| 100 | 10.6 ± 3.8 | 33 |

The data indicate that racemic menthol propylene-carbonate inhibited microvessel formation in rat aortic ring assay in a dose dependent manner.

Example 3 describes the results of a cell cytotoxicty assay performed to evaluate the ctytoxic effect of racemic menthol propyleneglycol-carbonate on normal cells.

### Example 3

5×10³ Ha-CaT cells were plated in each well of 96 well plates and racemic menthol propyleneglycol-carbonate was added at various concentrations. The plates were incubated for another 48 hours and the viable cells were measured by XTT Cell Proliferation Kit (Roche). More than 70% of Ha-CaT cells were viable even at high concentration of 100 μM. The results of this experiment are shown in Table III.

TABLE III

| Concentration (uM) | Cell Viability (%) |
|---|---|
| 0.1 | 100 |
| 1.0 | 100 |
| 5.0 | 96.5 |
| 10.0 | 96.3 |
| 50.0 | 81.8 |
| 100 | 70.4 |

These data show that racemic menthol propyleneglycol-carbonate is non-toxic against normal cells.

Example 4 shows the results of an experiment conducted to determine the effect of racemic menthol propyleneglycol carbonate on HUVE cell proliferation.

### Example 4

HUVE (Human Umbilical Vein Endothelial) cells were isolated from human umbilical cord veins by a method of Jaffe et al. (Jaffe, E. A., Nachman, R. L., Becker, C. G., and Minick, C. R. Culture of Human endothelial cells derived from umbilical veins. J. Clin. Invest. 52: 2745-2756, 1973). HUVE cells were confirmed by immunostaining with antibody against factor VIII. The cells were grown in M199 medium (Gibco BRL, Grand Island, N.Y.) supplemented with 10% fetal bovine serum, 100 units/mL penicillin, 100 μg/mL streptomycin, 50 μg/mL endothelial cell growth supplement, and 5 units/mL heparin at 37° C. in an atmosphere of 5% CO₂-95% air.

1×10⁴ HUVE cells were plated in each well of 96 well plates and five different concentrations ranging from 1 to 100 of racemic menthol propyleneglycol-carbonate were tested in the presence of bFGF used as maximum proliferation control. Cells were cultured for an additional 48 hours, and relative cell numbers in each well were determined by XTT Cell Proliferation Kit (Roche).

The results are presented in Table IV.

| | No | bFGF | MPC-r* | MPC-r | MPC-r | MPC-r | MPC-r |
|---|---|---|---|---|---|---|---|
| | bFGF | only | 100 μM | 50 μM | 10 μM | 5 μM | 1 μM |
| Average | 0.2283 | 1.0830 | 0.9121 | 1.0621 | 1.0425 | 1.0305 | 1.0623 |
| Proliferation | 0.0 | 100.0 | 80.0 | 97.6 | 95.3 | 93.9 | 97.6 |
| (%) | | | | | | | |

*MPC-r = racemic menthol propyleneglycol-carbonate

As can be seen from the data in Table IV, 100 uM of menthol propyleneglycol-carbonate weakly inhibited HUVE cell proliferation by 20%. The results show that this compound does not have detrimental effects on the normal proliferation of endothelial cells.

The data set forth in the foregoing examples indicate that compounds of Formula I, above, are: (1) effective as insect repellents; (2) effective as anti-inflammatory agents when applied topically; and (3) effective as anti-angiogenesis agents.

While certain embodiments of the present invention have been described and/or exemplified above, various other embodiments will be apparent to those skilled in the art from the foregoing disclosure. The present invention is, therefore, not limited to the particular embodiments described and/or exemplified, but is capable of considerable variation and modification without departure from the scope of the appended claims.

What is claimed is:

1. A method for achieving an effect in a mammal, wherein the effect is an anti-inflammatory effect or an anti-angiogenic effect, said method comprising administering to said mammal, in an amount sufficient to produce said effect, a compound of the formula: 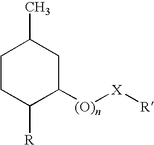
wherein R represents a straight or branched chain, substituted or unsubstituted lower alkyl radical, or a straight or branched chain, substituted or unsubstituted lower alkenyl radical;
X represents a carbonyl linking group (—C(═O)—) or a valence bond;
n is O or 1; and
R′ represents a radical selected from the group consisting of substituted or unsubstituted hydroxyalkyloxy and substituted or unsubstituted hydroxyalkyl, when n is 1; and R′ represents an alkylamine radical when n is O.

2. The method of claim 1 wherein the compound of Formula I is administered in the form of a composition, also including a suitable carrier, the amount of said compound being from about 1 wt % to about 80 wt % based on the total weight of said composition.

3. The method of claim 2, wherein the amount of said compound is from about 1 wt % to about 30 wt %, based on the total weight of said composition.

4. The method of claim 1, wherein the compound of Formula I is administered to produce an anti-inflammatory effect.

5. The method of claim 4, wherein the compound of Formula I is selected from the group consisting of menthoxy-propane-1,2-diol, N-ethyl-p-menthane-3-carboxyamide, menthol propyleneglycol-carbonate, isopulegol propyleneglycol-carbonate and menthyl-9-hydroxynonyl carbonate, said compound being in an enantiomerically pure form or in racemic form.

6. The method of claim 4, wherein the compound of Formula I is menthoxy-propane-1,2-diol.

7. The method of claim 4, wherein the compound of Formula I is racemic menthol propyleneglycol-carbonate.

8. The method of claim 4, wherein the compound of Formula I is isopulegol propyleneglycol-carbonate.

9. The method of claim 4, wherein the compound of Formula I is menthyl-9-hydroxynonyl carbonate.

10. The method of claim 1, wherein the compound of Formula I is administered to produce an anti-angiogenic effect.

11. The method of claim 10, wherein the compound of Formula I is racemic menthol propyleneglycol-carbonate.

12. The method of claim 1 wherein the compound of Formula I is administered systemically to said mammal.

13. The method of claim 12, wherein said compound is administered as a sustained release formulation.

14. The method of claim 1 wherein the compound of Formula I is administered topically to said mammal.

15. The method of claim 1, wherein the compound of Formula I is administered for achieving said effect in a human.
